(19) United States
(12) Patent Application Publication
(10) Pub. No.: US 2025/0306044 A1
(43) Pub. Date: Oct. 2, 2025

(54) HEMOLYSIS AND DERIVATIZATION REAGENTS AND METHODS FOR DETERMINING LACTONE ANALYTES

(71) Applicant: Roche Diagnostics Operations, Inc., Indianapolis, IN (US)

(72) Inventors: Tobias Stueckl, München (DE); Gaston Hubertus Maria Vondenhoff, München (DE)

(21) Appl. No.: 18/862,946

(22) PCT Filed: Apr. 28, 2023
(86) PCT No.: PCT/EP2023/061358

§ 371 Date: Nov. 4, 2024

(30) Foreign Application Priority Data
May 2, 2022 (EP) 22171213.6

(51) Int. Cl. G01N 33/94; G01N 30/06; G01N 30/88

(52) U.S. Cl. CPC G01N 33/9493; G01N 30/88; G01N 2030/067; G01N 2030/884

(57) ABSTRACT

The present invention relates to a method for determining a lactone analyte in a sample, comprising (i) contacting said sample with a derivatization reagent comprising a nucleophilic reagent; (ii) determining a nucleophilic reagent-derivative of said lactone analyte obtained in step (i), and (iii) determining said lactone analyte based on the determination of the nucleophilic reagent-derivative of said lactone analyte in step (ii). The present invention also relates to methods uses, compounds, kits and devices related thereto.

The present invention relates to a method for determining a lactone analyte in a sample, comprising (i) contacting said sample with a derivatization reagent comprising a nucleophilic reagent; (ii) determining a nucleophilic reagent-derivative of said lactone analyte obtained in step (i), and (iii) determining said lactone analyte based on the determination of the nucleophilic reagent-derivative of said lactone analyte in step (ii). The present invention also relates to methods, uses, compounds, kits and devices related thereto.

Everolimus (Eve), Sirolimus (Sir) and Tacrolimus (Tac) are macrolide compounds which are used in medicine as immunosuppressive drugs (ISD) after organ transplant. These drugs have a narrow therapeutic range, where lower levels are associated with insufficient therapy and eventually lead to rejection of the organ, and on the other hand, where higher levels are associated with toxicity to certain organs like liver and kidney. In addition, intra- and inter-patient variability in pharmacokinetic and pharmacodynamics parameters complicates a balanced dosage of the ISD. Therefore, therapeutic drug monitoring (TDM) of these drugs has become an indispensable adjunct for the effective treatment of transplant patients by controlling the therapy based on concentrations in the body rather than by dose alone. Since Everolimus, Sirolimus and Tacrolimus are highly bound to erythrocytes in the blood, the recommended matrix for the quantitation is whole blood.

For the determination of Eve, Sir and Tac in whole blood, a broad range of TDM tests are commercially available, typically as immunoassays for automated analyzers, but also as LC-MS/MS tests. One important aspect for tests running on automated in vitro diagnostics (IVD) platforms is that all the required reagents, such as immunoreagents, controls, calibrators and internal standards in case of LC-MS/MS, are ideally provided as stable and ready-to-use solutions. These solutions can be placed on-board of the instrument for a longer period of time, offering a workflow not needing user interactions. The use of reagents as lyophilisates is necessary when chemically instable reagents are involved, however, this hampers the workflow by requiring manual handling steps for reconstitution. Manual handling steps generate additional costs and also provide sources for errors. In addition, the reconstituted reagent typically shows short shelf-life and has to be replaced by freshly prepared solutions.

In contrast to solutions in organic solvents (e.g. methanol, acetonitrile), aqueous formulations of Eve, Sir and Tac are typically not stable for longer periods of time at storage temperatures which are used in lab refrigerators (2-8° C.) and on-board of analyzers (4-12° C.). It is known from literature (e.g. EP 2 402 350 A1) that the macrolides Eve, Sir and Tac are unstable in solution as a consequence of hydrolysis of their ester linkage, leading to a loss of biological activity. Thus, aqueous formulations of Eve, Sir and Tac, which are free of organic solvents, including blood-based samples, are typically stored at very low temperatures ranging from −20 to −80° C. in order to keep them stable and usable in IVD for longer periods of time; otherwise the solutions are only stable for a short time period (1-6 weeks at 2-8° C.).

To date, both internal standards (ISTDs) and calibrators for immunomodulatory macrolides need to be produced, shipped and stored under utmost care. There are several problems associated with this: Firstly, performing TDM from whole blood requires extensive (manual) sample preparation to extract the analytes of interest from the blood prior to quantitation of these compounds. This effort is time, labor and materials intensive. Furthermore, a risk of introducing errors exist with every step that needs to be performed during sample preparation.

Further, calibration needs to be done with a known concentration of the analytes that one aims to quantify. With an instable compound in the calibration solution, this is a challenge. Furthermore, the ISTD concentration needs to be the same during calibration and sample measurement. An ISTD concentration changing over time affects the result directly and may lead to overestimation of the result in case the ISTD concentration is higher during calibration than during sample measurement. Thus, with the available methodology, a risk of biased results as well as consequently incorrect patient dosing may result. Currently, frequent re-calibration is used to ascertain that the ISTD concentration is similar during calibration and sample measurement. However, this reduces the throughput of an analyzer. Moreover, a decreasing ISTD concentration compromises the usability (i.e. shelf life) of a solution. With that, costs are high and high reagent turnover is not environmentally friendly.

There is, thus, a need in the art for improved means and methods for determining ketone analytes, in particular immunomodulatory macrolides. This problem is solved by the means and methods disclosed herein.

In accordance, the present invention relates to a method for determining a lactone analyte in a sample, comprising
(i) contacting said sample with a derivatization reagent comprising a nucleophilic reagent;
(ii) determining a nucleophilic reagent-derivative of said lactone analyte obtained in step (i), and
(iii) determining said lactone analyte based on the determination of the nucleophilic reagent-derivative of said lactone analyte in step (ii).

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” in an embodiment refer to “comprising one or more”, i.e. are equivalent to “comprising at least one”. In accordance, expressions relating to one item of a plurality, unless otherwise indicated, in an embodiment relate to at least one such item, in a further embodiment a plurality thereof; thus, e.g. identifying “a cell” relates to identifying at least one cell, in an embodiment to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, in an embodiment, are in vitro methods. The method steps may, in principle, be performed in any arbitrary sequence deemed suitable by the skilled person, but in an embodiment are performed in the indicated sequence; also, one or more, in an embodiment all, of said steps may be assisted or performed by automated equipment. Moreover, the methods may comprise steps in addition to those explicitly mentioned above.

Method steps may be performed, in principle, under environmental conditions deemed appropriate by the skilled person, e.g. under standard conditions. In particular the method steps may be performed at a temperature, selected for each method step independently, of from 0° C. to 80° C., in an embodiment 10° C. to 60° C., in a further embodiment of from 15° C. to 50° C., in a further embodiment of from 20° C. to 40° C. Also, pressure may in an embodiment be ambient pressure. The pH may be the pH of the sample; the pH may, however, also be adjusted e.g. to a neutral pH, e.g. during step (i), and/or to acidic during step (ii); thus, during all or part of step step (ii), the pH may be in the range of from 1 to 6, in an embodiment of from 2 to 5, in a further embodiment of from 3 to 4. Thus, during all or part of step (ii), the pH may be about 1, about 2, about 3, about 4, about 5, or about 6.

As used herein, the term “standard conditions”, if not otherwise noted, relates to IUPAC standard ambient temperature and pressure (SATP) conditions, i.e. in an embodiment, a temperature of 25° C. and an absolute pressure of 100 kPa; also in an embodiment, standard conditions include a pH of 7. Moreover, if not otherwise indicated, the term “about” relates to the indicated value with the commonly accepted technical precision in the relevant field, in an embodiment relates to the indicated value±20%, in a further embodiment±10%, in a further embodiment±5%. Further, the term “essentially” indicates that deviations having influence on the indicated result or use are absent. i.e. potential deviations do not cause the indicated result to deviate by more than ±20%, in a further embodiment ±10%, in a further embodiment±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. In an embodiment, a composition consisting essentially of a set of components will comprise less than 5% by weight, in a further embodiment less than 3% by weight, in a further embodiment less than 1% by weight, in a further embodiment less than 0.1% by weight of non-specified component(s).

The method for determining a lactone analyte is an in vitro method. Methods for determining a lactone analyte in a sample are, in principle, known to the skilled person. Moreover, the method may comprise steps in addition to those explicitly mentioned above. For example, further steps may relate, e.g., to providing one or more samples for (i), or contacting said sample with further compounds, in particular an internal standard before step (ii), in an embodiment before step (i). Moreover, one or more of said steps may be performed or assisted by automated equipment.

The term “determining” is understood by the skilled person. In an embodiment, as used herein, the term refers to determining at least one characteristic feature of an analyte to be determined. Characteristic features in accordance with the present invention are features which characterize the physical and/or chemical properties including biochemical properties of an analyte. Such properties include, e.g., molecular weight, viscosity, density, electrical charge, spin, optical activity, color, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of an analyte by standard operations, e.g., mathematical calculations such as multiplication, division, logarithmic calculus, and/or standardization. In an embodiment, the at least one characteristic feature allows the determination and/or chemical identification of the analyte and its amount. Accordingly, the characteristic value, in an embodiment, also comprises information relating to the abundance of the analyte from which the characteristic value is derived. For example, a characteristic value of an analyte may be a peak in a mass spectrum. Such a peak contains characteristic information of the analyte, i.e. the m/z information, as well as an intensity value being related to the abundance of the said analyte (i.e. its amount) in the extract.

As used herein, the term “analyte” relates to a chemical compound present in a sample of a subject, in an embodiment, in a body fluid. In an embodiment, the analyte is a small molecule, i.e., in an embodiment, the analyte is not a biological macromolecule. The analyte is an organic molecule, in an embodiment a molecule comprising at least one carbon-carbon bond. In an embodiment, the analyte is a molecule of the subject's metabolism. In a further embodiment, the analyte is a compound administered to said subject, e.g. in medical treatment, including prophylactic treatment. Also in an embodiment, the analyte is a low molecular weight chemical compound, in an embodiment with a molecular mass of at most 5000 Da, in an embodiment at most 2000 Da, in a further embodiment at most 1500 Da. In a further embodiment, the analyte has a molecular mass of from 50 Da to 5000 Da, in a further embodiment of from 100 Da to 2000 Da, in a further embodiment of from 250 to 1500 Da.

As referred to herein, the analyte is a lactone analyte. The term “lactone analyte”, as used herein, relates to a chemical molecule comprising at least one lactone group, i.e. an intramolecular ester group. Corresponding compounds are known to the skilled person. In an embodiment, the lactone analyte is a macrocyclic lactone comprising an at least eight-membered lactone ring, in an embodiment an at least twelve-membered lactone ring. In an embodiment, the lactone analyte comprises an at least 5-membered lactone ring; in a further embodiment, the lactone analyte comprises a lactone ring comprises of from 5 to 50 ring atoms, in an embodiment of from 12 to 40 ring atoms, in a further embodiment of from 15 to 35 ring atoms. In an embodiment, the lactone analyte comprises a 20, 21, 22, 23, 24, 25, 26, 27, 28, 29, 30, 31, 32, 33, 34, or 35-membered lactone ring. In an embodiment, the lactone analyte is a macrolide, in a further embodiment is an immunomodulatory macrolide, an antibiotic macrolide, an antimycotic macrolide, or a mycotoxin macrolide. In an embodiment, the lactone analyte is an immunomodulatory macrolide.

The term “immunomodulatory macrolide”, as used herein, relates to a lactone analyte, in an embodiment a macrolide, as specified herein above having the property of modulating the immune response of a subject, in an embodiment of a mammal, in particular a human. In an embodiment, said modulation is a suppression; thus, in an embodiment, the immunomodulatory macrolide is an immune-suppressive compound of the macrolide class of compounds, i.e. is a macrolide immunosuppressant. In an embodiment, the immunomodulatory macrolide is a compound inhibiting T lymphocyte activation; means and methods for determining such activity are known to the skilled person. As will be understood by the skilled person, the immunomodulatory effect of an immunomodulatory macrolide may be direct, i.e. by the compound itself modulating an activity of a component of the immune system, or may indirect, e.g. by a metabolite of the immunomodulatory macrolide modulating an activity of a component of the immune system. In an embodiment, the immunomodulatory macrolide is comprised in a sample, in particular a sample of a body fluid, at a concentration of from 1 ng/ml to 15 μg/ml, in an embodiment of from 5 ng/ml to 1 μg/ml, in a further embodiment of from 7.5 ng/ml to 500 ng/ml, in a further embodiment of from 10 ng/ml to 250 ng/ml.

In an embodiment, the lactone analyte is Everolimus ((1R,9S,12S,15R,16E,18R,19R,21R,23S,24E,26E,28E,30S,32S,35R)-1,18-dihydroxy-12-[(2R)-1-[(1S,3R, 4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}] hexatriaconta-16,24,26,28-tetraene-2,3,10,14,20-pentone, CAS-number 159351-69-6), Sirolimus ((1R,9S,12S,15R,16E,18R,19R,21R,23S,24E,26E,28E,30S,32S,35R)-1,18-dihydroxy-12-{(2R)-1-[(1S,3R,4R)-4-hydroxy-3-methoxycyclohexyl]-2-propanyl}-19,30-dimethoxy-15,17,21,23,29,35-hexamethyl-11,36-dioxa-4-azatricyclo[30.3.1.04,9]hexatriaconta-16,24,26,28-tetraene-2,3,10,14,20-pentone, CAS number 53123-88-9, Tacrolimus ((1R,9S,12S,13R,14S,17R,18E,21S,23S,24R,25S,27R)-1,14-dihydroxy-12-[(1E)-1-[(1R,3R,4R)-4-hydroxy-3-methoxycyclohexyl]prop-1-en-2-yl]-23,25-dimethoxy-13,19,21,27-tetramethyl-17-(prop-2-en-1-yl)-11,28-dioxa-4-azatricyclo [22.3.1.0^{4,9}]octacos-18-ene-2,3,10,16-tetrone, CAS number 104987-11-3), Pimecrolimus ((1R,9S,12S,13R,14S,17R,18E,21S,23S,24R,25S,27R)-12-[(1E)-1-[(1R,3R,4S)-4-chloro-3-methoxycyclohexyl]prop-1-en-2-yl]-17-ethyl-1,14-dihydroxy-23,25-dimethoxy-13,19,21,27-tetramethyl-11,28-dioxa-4-azatricyclo[22.3.1.0^{4,9}]octacos-18-ene-2,3,10,16-tetrone, CAS number 137071-32-0), or Temsirolimus ((1R,2R,4S)-4-[(2R)-2-[(1R,9S,12S,15R,16E,18R,19R,21R,23S,24E,26E,28E,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl 3-hydroxy-2-(hydroxymethyl)-2-methylpropanoate, CAS number 162635-04-3). In an embodiment, the lactone analyte is selected from Everolimus, Sirolimus, Tacrolimus, Pimecrolimus and Temsirolimus, in an embodiment is selected from Everolimus, Sirolimus, and Tacrolimus, in a further embodiment is Everolimus, in an embodiment is Sirolimus, in an embodiment is Tacrolimus.

The term “sample”, as used herein, refers to any composition of matter suspected or known to comprise at least one lactone analyte. In an embodiment, the sample is a sample of a subject, in an embodiment of a patient; in an embodiment, the sample is an isolated sample from a subject.

Thus, in an embodiment, a sample is a sample of a body fluid, in an embodiment, blood, plasma, serum, saliva or urine, or a sample derived by lavage from tissues or organs, e.g. from the respiratory tract. In a further embodiment, the sample is a blood, plasma, serum or urine sample. In a further embodiment, the sample is a blood or plasma sample or is a serum or plasma sample, in a further embodiment is a blood sample. In an embodiment, in case the sample is a blood sample, the method of the present invention comprises a further step of obtaining a serum or plasma sample from said blood sample, comprises treating said sample with a release agent, or comprises hemolysing said sample. In an embodiment, the sample is a citrate blood sample, a heparin blood sample, or an EDTA blood sample. In a further embodiment, the sample is an EDTA blood sample. Biological samples can be derived from a subject as specified elsewhere herein. Techniques for obtaining the aforementioned different types of biological samples are well known in the art. For example, blood samples may be obtained by blood taking, e.g. by puncturing an arterial and/or a venous blood vessel. In an embodiment, the sample is a sample of cells, tissue, or an organ obtained from a subject. In an embodiment, in case said sample is an organ, said subject is not a human. Solid samples can be obtained by well known techniques including, in an embodiment, scrapes, swabs or biopsies appropriate regions of a body. As is known to the skilled person, such samples can be obtained by use of brushes, (cotton) swabs, spatulae, rinse/wash fluids, punch biopsy devices, puncture of cavities with needles or surgical instrumentation.

The term “contacting”; as used herein, is understood by the skilled person. In an embodiment, the term relates at least to bringing a sample and/or an analyte of the present invention into physical contact with a derivatization reagent, thereby allowing the sample and/or analyte to interact with the nucleophilic reagent.

The term “nucleophilic reagent” is understood by the skilled person to relate to a chemical species that donates an electron pair to form a chemical bond. Nucleophiles that exist in a water medium include but are not limited to —NH₂, —NHR, —OH, —SH, —Se⁻; (R′, R″, R′″)P, N₃, RCOOH, F⁻, Cl⁻, Br⁻, and I⁻. A nucleophilic reagent, in an embodiment, comprises a moiety, carrying an orbital that serves as the highest occupied molecular orbital (HOMO) that is able to attack the lowest unoccupied molecular orbital (LUMO) of the lactone analyte, thereby forming a new molecule comprised of the formerly nucleophilic unit and the analyte moiety. In an embodiment, the term includes amines, alcohols, thiols, and selenols. In an embodiment, the nucleophilic reagent is an amine, in a further embodiment is a primary or secondary amine. In an embodiment, the nucleophilic reagent is a primary or secondary alkyl amine, in a further embodiment a C₁ to C₈ alkyl primary or secondary amine. In a further embodiment, the nucleophilic reagent is a primary amine, in a further embodiment a primary alkyl amine, in a further embodiment a C₁ to C₈ alkyl primary amine. In a further embodiment, the nucleophilic reagent is butylamine, propylamine, pentylamine, or hexylamine, in an embodiment n-butylamine, n-propylamine, n-pentylamine, or n-hexylamine. In an embodiment, the nucleophilic reagent is an n-alkyl primary amine, in an embodiment a C1 to C8 n-alkyl primary amine, in a further embodiment a butylamine, in a further embodiment is n-butylamine (CAS NO: 109-73-9).

The term “derivatization reagent”, as used herein, relates to any composition comprising at least one nucleophilic reagent as specified herein above. In an embodiment, the derivatization reagent is a liquid composition, in an embodiment comprising at least one solvent in addition to the nucleophilic reagent, such as methanol, acetonitrile, and the like. The derivatization reagent may comprise further compounds, such as a buffer, one or more enzymes, in particular a hydrolase such as a DNase, RNase, and/or a protease, at least one salt, and any other compound deemed appropriate by the skilled person. In a further embodiment, the derivatization reagent only comprises the nucleophilic reagent, i.e. consists of or essentially consists of the nucleophilic reagent.

The method for determining a lactone analyte in a sample as specified herein comprises step (i) contacting said sample with a derivatization reagent comprising a nucleophilic reagent. In an embodiment, the concentration of the nucleophilic reagent in step (i) is of from 0.1 M to 10 M, in an embodiment is of from 0.5 M to 1.5 M, in a further embodiment is about 1 M, in an embodiment is 1 M. In a further embodiment, the derivatization reagent comprising the nucleophilic reagent at a concentration of from 1 M to 10 M is contacted with said sample at a ratio of from 1:5 to 10:1, in an embodiment of from 1:2 to 5:1 in step (i). In a further embodiment, the pH during the contacting in step (i) is of from 4 to 10, in an embodiment of from 5 to 9, in a further embodiment of from 6 to 8. The duration of step (a) may, in principle, be any time deemed appropriate by the skilled person. In an embodiment, the sample is contacted with the derivatization reagent for at least 10s, in a further embodiment for at least 1 minute, in a further embodiment at least 2 minutes; also in an embodiment, the sample is contacted with the derivatization reagent for less than 5 minutes, in an embodiment less than 2 minutes, in a further embodiment less than 1 minute. Thus, the sample may be contacted with the derivatization reagent for of from 10 s to 5 min, in an embodiment of from 30 s to 4 min, in a further embodiment of from 1 min to 3 min. However, since the lactone analyte is essentially stable after contacting with the derivatization reagent, also longer contacting times are envisaged for up to 30 min, in an embodiment up to 1 h, in a further embodiment up to 12 h, in a further embodiment up to 24 h, in a further embodiment up to 3 days, in a further embodiment up to 1 week.

The sample may, in an embodiment, be pre-treated before use in the method as specified herein. Said pre-treatment may include treatments required to release or separate the compounds comprised in the sample or to remove excessive material or waste. Suitable techniques comprise centrifugation, extraction, fractioning, ultrafiltration, protein precipitation followed by filtration and purification and/or enrichment of compounds. Thus, the method may in particular comprise removing insoluble sample constituents before step (ii), in an embodiment by centrifugation and/or filtration. Also, optionally, the method for determining a lactone analyte further comprises enriching the lactone analyte and/or the nucleophilic reagent-derivative of said lactone analyte before step (ii), in an embodiment by binding to a solid surface, e.g. by an immunological method. In particular in case the sample is a sample comprising cells, the method may further comprise a step of lysing cells preceding step (ii), e.g. by contacting the sample with a lysis reagent. As indicated herein above, pre-treatment may, e.g. be treating a blood sample with a release agent, in an embodiment an organic solvent, in particular methanol or acetonitrile. Moreover, other pre-treatments may be carried out in order to provide the analyte or analytes in a form or concentration suitable for analysis. Pre-treated samples as described herein are also comprised by the term “sample” as used herein. Optionally, pre-treatment steps, e.g. the step of lysing cells, may also be performed concomitant to step (i). In an embodiment, in particular in case the sample is a sample comprising cells, in an embodiment a blood sample or a blood-derived sample, said sample is directly contacted with said nucleophilic reagent; in an embodiment, in such case, said contacting with a nucleophilic reagent is the only sample treatment step before step (ii), i.e. step (i) consists of contacting the sample with the nucleophilic reagent and is followed directly by step (ii), without any intervening steps. Thus, in an embodiment, the method does not comprise a step of removing insoluble sample constituents in and between steps (i) and step (ii). In accordance, the derivatization reagent, in an embodiment the nucleophilic reagent, may be the release agent. Step (i) in an embodiment is performed at most 12 h, in an embodiment at most 3 h, in a further embodiment at most 1 h, in a further embodiment at most 10 min, after the sample is obtained. As the skilled person will understand, the time until step (i) may be shortened further by using e.g. blood sampling vials already comprising an appropriate amount of derivatization reagent. In an embodiment, the method comprises step (i) consisting of contacting the sample with the nucleophilic reagent followed directly by step (ii) comprising, in an embodiment consisting of, addition of an internal standard, optionally enriching the nucleophilic reagent-derivative of said lactone analyte, e.g. as specified herein above, and determining the nucleophilic reagent-derivative of said lactone analyte by MS, in an embodiment LC-Ms or GC-MS. As specified elsewhere herein, the pH in an embodiment is acidic during MS and LC or GC, in an embodiment is acidic as specified herein above after or starting with optional addition of an internal standard and/or during enrichment; said acidification is particularly envisaged in case the nucleophilic reagent is an amine, in an embodiment a primary amine.

The method for determining a lactone analyte in a sample as specified herein further comprises step (ii) determining a nucleophilic reagent-derivative of said lactone analyte obtained in step (i). As indicated herein above, determining includes each and every method of determining at least one characteristic feature of an analyte to be determined deemed appropriate by the skilled person. In an embodiment, determining step (ii) comprises quantitatively determining a parameter directly proportional to the concentration of the lactone analyte and/or the nucleophilic reagent-derivative of said lactone analyte, in an embodiment to the nucleophilic reagent-derivative of said lactone analyte. In an embodiment, step (ii) comprises quantitatively determining a parameter specifically and directly proportional to the concentration of the nucleophilic reagent-derivative of said lactone analyte. Thus, determining a nucleophilic reagent-derivative of the lactone analyte may be performed e.g. by a mass spectrometry method, an immunological method, a chromatographic method, and the like. Immunological methods are known to the skilled person, e.g. ELISA methods, ECL methods, and the like; in such methods, specifically recognizing the nucleophilic reagent-derivative of said lactone analyte obtained in step (i) may be used. In an embodiment, step (ii) comprises separating said nucleophilic reagent-derivative of said lactone analyte from sample matrix constituents, in an embodiment by chromatography, in a further embodiment liquid chromatography (LC) or gas chromatography (GC). As will be understood by the skilled person, in particular in low-complexity samples, determining the nucleophilic reagent-derivative of the lactone analyte may already be possible by a spectroscopic step following the chromatographic step, e.g. UV or UV/VIS spectroscopy, which may in an embodiment be coupled in-line to the outlet of a chromatography column. As will be also understood by the skilled person, the determining step may be aided by an appropriate selection of the nucleophilic reagent to provide e.g. appropriate spectroscopic properties. In an embodiment, step (ii) comprises mass spectrometry (MS), in an embodiment tandem MS, in a further embodiment comprises quadrupole MS. MS may be performed directly on a sample according to step (i), in particular for low-complexiy samples; the nucleophilic reagent-derivative of the lactone analyte may, in an embodiment, be enriched, e.g. by binding to a solid surface and/or by chromatography. Thus, in an embodiment, MS is coupled to at least one of the aforesaid steps of separating the nucleophilic reagent-derivative of the lactone analyte from sample matrix constituents, in particular binding to a solid surface and/or chromatography. Thus, in an embodiment, step (ii) comprises determining said nucleophilic reagent-derivative of said lactone analyte by a method comprising, in an embodiment consisting of, LC-MS or GC-MS, in an embodiment LC-MS, in a further embodiment LC-MS/MS.

The method for determining a lactone analyte in a sample as specified herein comprises step (iii) determining said lactone analyte based on the determination of the nucleophilic reagent-derivative of said lactone analyte in step (ii). As will be understood by the skilled person, the specific method of determining the lactone analyte based on the determination of the nucleophilic reagent-derivative of said lactone analyte in step (iii) will depend on a variety of parameters, such as the type of determination desired (quantitative or semiquantitative), the method of determining used in step (ii), among others. E.g. in case a chromatography/UV-spectroscopy method is used, determination may already be possible based on the peak obtained for the nucleophilic reagent-derivative of the lactone analyte, e.g. taking into account the molar absorption coefficient of the nucleophilic reagent-derivative of the lactone analyte. In an embodiment, the method comprises measurement of at least one, in an embodiment at least two, in an embodiment at least three, in a further embodiment at least four, in a further embodiment at least five, calibration samples and determination of the lactone analyte by comparing the value of the parameter directly proportional to the concentration of the lactone analyte and/or the nucleophilic reagent-derivative of the lactone analyte obtained in step (ii) to a value of a corresponding parameter obtained for at least one calibration sample. Thus, in an embodiment, step (ii) further comprises obtaining a calibration curve based on at least two calibration samples comprising two pre-determined and non-identical concentrations of said nucleophilic reagent-derivative of said lactone analyte. In case an internal standard is included, the value of the parameter directly proportional to the concentration of the lactone analyte and/or the nucleophilic reagent-derivative of the lactone analyte obtained in step (ii) may also be compared to a value of a corresponding parameter obtained for said internal standard. From the above, in an embodiment, an amount or concentration of the lactone analyte in the sample may be calculated.

Optionally, the method for determining a lactone analyte in a sample as specified herein further comprises adding an internal standard to said sample before step (ii), in an embodiment before step (i). Said internal standard comprises, in an embodiment is, an isotope-labeled derivative of the lactone analyte and/or of the nucleophilic reagent-derivative of said lactone analyte, in an embodiment of the nucleophilic reagent-derivative of said lactone analyte, in an embodiment as specified herein below. In such case, determining step (ii) in an embodiment further comprises quantitatively determining a parameter directly proportional to the concentration of the isotope-labeled derivative of the lactone analyte and/or of the nucleophilic reagent-derivative of said lactone analyte. The internal standard may in particular comprise an isotope-labeled derivative of the lactone analyte and/or of the a nucleophilic reagent-derivative of said lactone analyte as specified herein below, in particular an isotope-labeled butylamide derivative of the lactone analyte, such as an isotope-labeled n-butylamide derivative of Everolimus, Sirolimus and/or Tacrolimus, in an embodiment Everolimus-butylamide-d9, Sirolimus-butylamide-d9, and/or Tacrolimus-butylamide-d9. The internal standard may, however, also be a derivative of the nucleophilic reagent-derivative of said lactone analyte sufficiently similar to the nucleophilic reagent-derivative of said lactone analyte to serve as an internal standard; thus, in such case, the internal standard in an embodiment has chromatographic properties essentially similar to those of the nucleophilic reagent-derivative of said lactone analyte, but provides for at least one non-identical ion in MS. Thus, the internal standard may e.g. be a derivative of the nucleophilic reagent-derivative of said lactone analyte comprising an additional or lacking a CH₂ group, a methyl group, a halogen group, or the like. Thus, an internal standard of a lactone analyte contacted with butylamine may, e.g. be a propylamine or pentylamine derivative of said lactone analyte.

Advantageously, it was found in the work underlying the present invention that lactone analytes, in particular unstable lactone analytes such as macrolides, can be stabilized before analysis by derivatization with a nucleophilic reagent. The derivatives obtained were found to be much more stable than the lactone analytes, allowing more reliable analysis. Moreover, it was found that nucleophilic reagents can be used as lysis reagents, allowing almost instant derivatization of lactone analytes and at the same time enabling lysis of cells, essentially obviating the need for additional working steps for removing insoluble compounds from the sample before analysis.

The definitions made above apply mutatis mutandis to the following. Additional definitions and explanations made further below also apply for all embodiments described in this specification mutatis mutandis.

The present invention further relates to a method of lysing cells in a biological sample, comprising contacting said biological sample with a nucleophilic reagent, as specified herein.

In the method of lysing cells, the sample, in an embodiment, is a biological sample; the sample may in particular be a liquid sample comprising cells, in particular blood, plasma, serum, saliva or urine, or a sample derived by lavage from tissues or organs, e.g. from the respiratory tract, all as specified herein above. In a further embodiment, the sample is a blood, plasma, or serum or urine sample, in a further embodiment is a blood sample.

In the method of lysing cells, the nucleophilic reagent, in an embodiment, is an amine, an alcohol, a thiol, or a selenol, in an embodiment is a primary amine, in an embodiment a primary alkyl amine, in a further embodiment a C1 to C8 alkyl primary amine, in an embodiment an n-alkyl primary amine, in a further embodiment a C1 to C8 n-alkyl primary amine, in a further embodiment is a butylamine, propylamine, pentylamine, or hexylamine, in an embodiment n-butylamine, n-propylamine, n-pentylamine, or n-hexylamine, in a further embodiment is n-butylamine (CAS NO: 109-73-9). Suitable concentrations of the nucleophilic agent have been specified herein above. In an embodiment, after lysing cells according to the method as specified herein, no precipitate is formed, i.e., in an embodiment, a clear lysate is obtained.

The present invention also relates to uses of a nucleophilic reagent for determining a lactone analyte in a sample and/or for lysing cells in a biological sample. Further, the present invention relates to a nucleophilic reagent-derivative of an immunomodulatory macrolide.

Immunomodulatory macrolides and nucleophilic reagents have been described herein above. In an embodiment, the nucleophilic reagent-derivative of an immunomodulatory macrolide has a structure as shown in one of Formulas (I) to (IX):

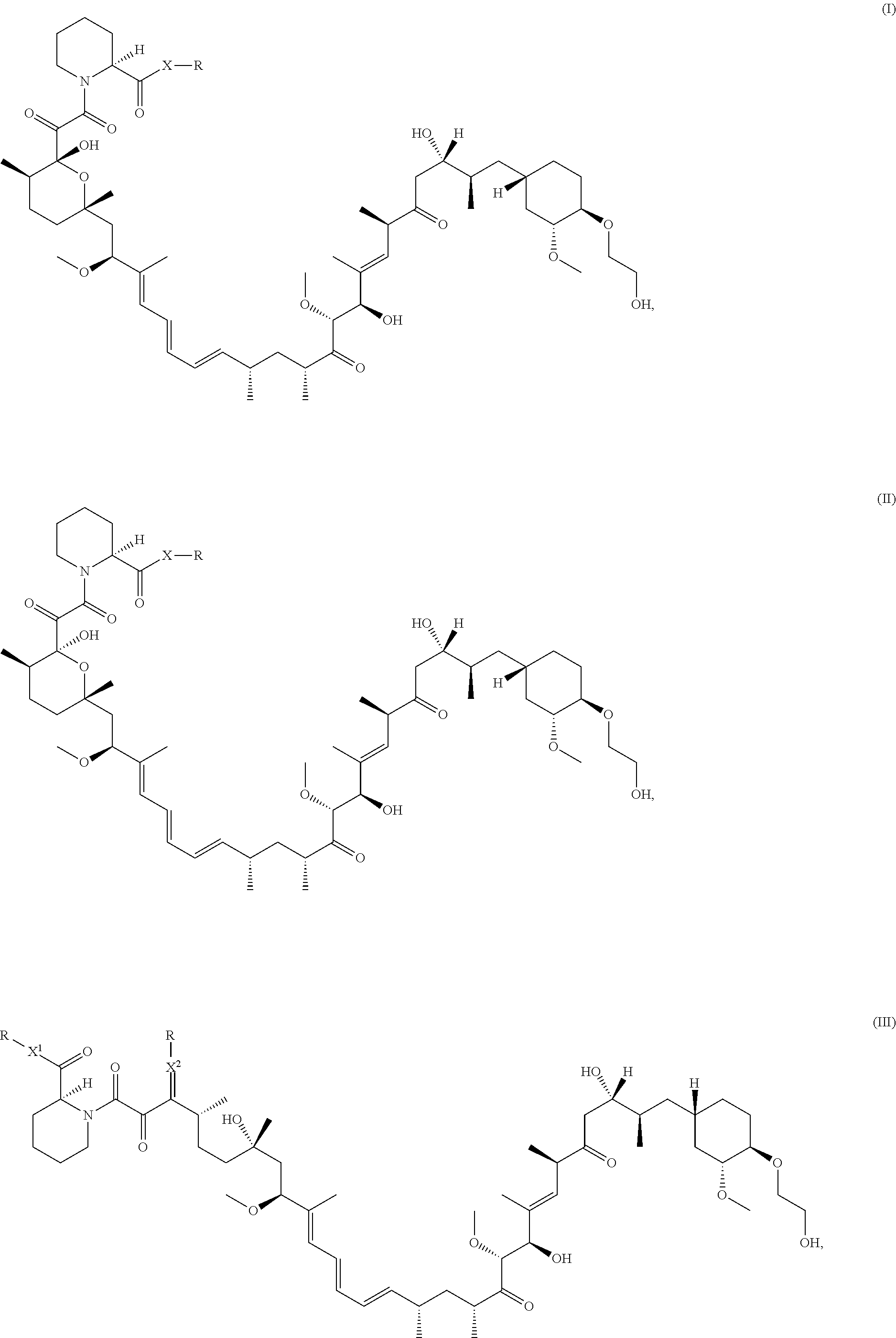

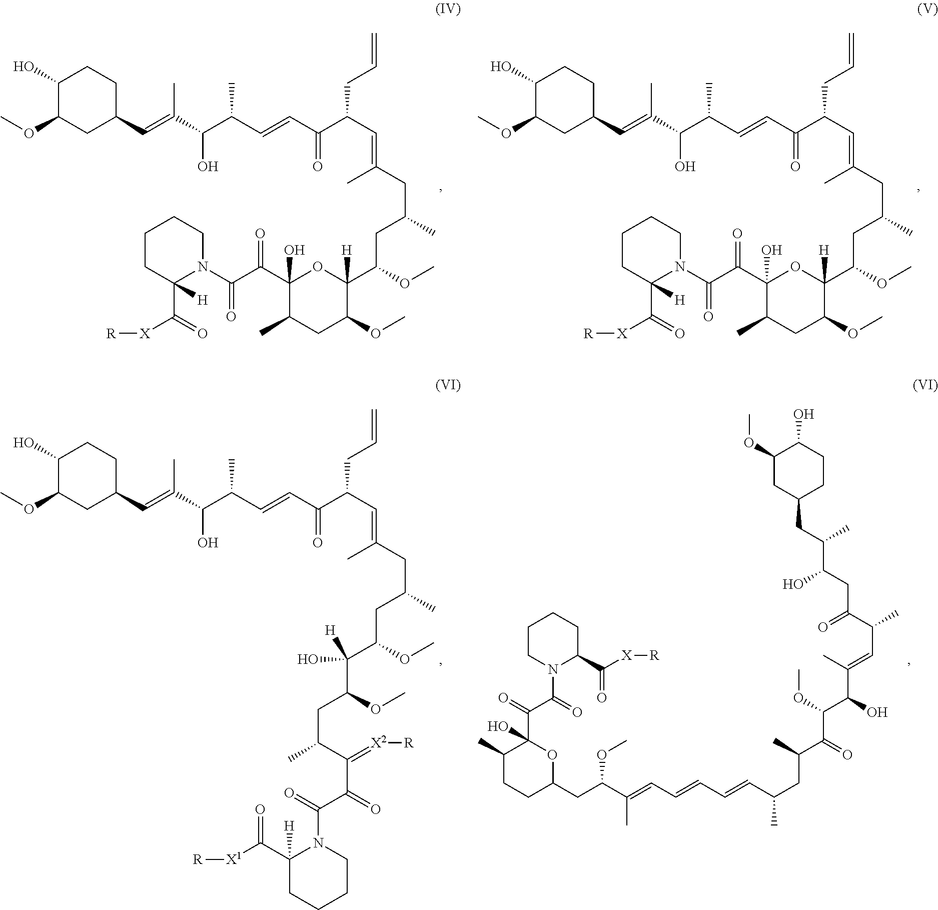

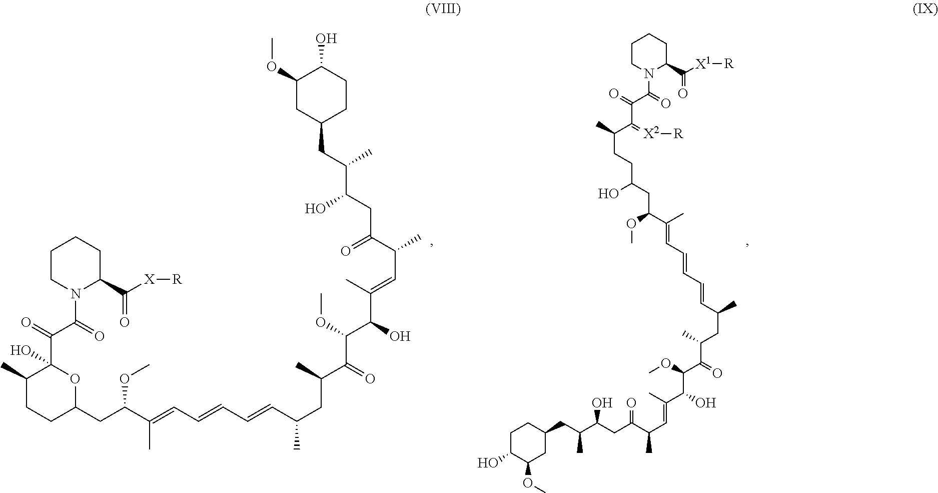

wherein in Formulas (I), (II), (IV), (V), (VII), and (VIII)
X is selected from NR^{a}, O, S, and Se, wherein R^{a} is selected from H and alkyl, in an embodiment from H and C₁ to C₈ alkyl and
R is selected from alkyl, in an embodiment from H and C₁ to C₈ alkyl, in an embodiment is butyl, propyl, pentyl, or hexyl;
and wherein in Formulas (III), (VI), and (IX)
X¹ is NR^{a} wherein R^{a} is selected from H and alkyl, in an embodiment from H and C₁ to C₈ alkyl,
X² is N⁺R^{a}, wherein R^{a} is selected from alkyl, in an embodiment from H and C₁ to C₈ alkyl; or is N; and
R is selected from alkyl, in an embodiment from H and C₁ to C₈ alkyl, in an embodiment is butyl, propyl, pentyl, or hexyl.

In an embodiment, in Formulas (I), (II), (IV), (V), (VII), and (VIII), X is NH, in a further embodiment R is butyl, in a further embodiment X is NH and R is butyl, in an embodiment n-butyl. In a further embodiment, in Formulas (III), (VI), and (IX) X¹ is NH and X² is N; in a further embodiment R is butyl, in a further embodiment X¹ is NH, X² is N, and R is butyl, in an embodiment n-butyl.

In an embodiment, the immunomodulatory macrolide and/or the nucleophilic reagent side chain is/are isotopically labeled, in an embodiment the nucleophilic reagent side chain is isotopically labelled. The term “isotopically labeled” is understood by the skilled person. In an embodiment, an isotopically labelled compound is a compound in which at least one atom was replaced by an isotope having a different mass. Thus, the isotopically labelled compound, i.e. the immunomodulatory macrolide, the nucleophilic reagent side chain, and/or the nucleophilic reagent-derivative of the lactone analyte is/are isotopologues of the corresponding compounds. In an embodiment, the isotopic label is selected such that the isotopically labelled compound has a mass increased by at least 3 u, in an embodiment at least 4 u compared to the non-labeled compound.

The term “isotopologue” is used herein in its conventional meaning to relate to a compound or group of compounds differing from a comparison compound only in its or their isotopic composition. Thus, the ions determined, e.g. in an MS method, for the isotopologues in an embodiment differ only in their isotope composition, in a further embodiment, the ions determined for the isotopologues and the analyte differ only in their isotope composition. As the skilled person is aware of, stable isomers, and therefore isotopologues, may be present in a preparation of a chemical compound in a statistical manner, caused e.g. by the natural distribution of isotopes. E.g. any carbon atom in a chemical molecule has a probability of about 1% of being ¹³C, unless artificially enriched or depleted. In view of the above, in an embodiment, the isotopologue is structurally identical to the analyte, but comprises at least one isotope label, i.e. in an embodiment at least one position in which an atom is replaced by an isotope thereof. For the avoidance of doubt, as used herein, the term isotopologue refers a population of molecules in which, compared to an analyte, the same number of atoms was replaced by the same isotope; this may be a population of molecules in which the same atom(s) in the same positions(s) were replaced, in which case the isotopologue may also be referred to as “specific isotopologue”; the isotopologue may, however, also be population of molecules in which the same number of the same atoms was replaced by the same number of the same isotopes, in which case the isotopologue may also be referred to as “mixed isotopologue”. In an embodiment, the isotopologue is a specific isotopologue.

In an embodiment, the isotope is a stable isotope. In an embodiment, the atom replaced is a carbon atom and the isotope is ¹³C; in a further embodiment, the atom replaced is nitrogen and the isotope is ¹⁵N; in a further embodiment, the atom replaced is hydrogen and the isotope is ²H; in a further embodiment, the atom replaced is oxygen and the isotope is ¹⁷O or ¹⁸O, in an embodiment is ¹⁸O; in a further embodiment, the atom replaced is sulfur and the isotope is ³³S or ³⁴S. In an embodiment, at least two, in a further embodiment at least three, in a further embodiment at least four, in a further embodiment at least five atoms in the analyte structure are replaced by their isotopes; in such case, in an embodiment only atoms of the same element are replaced and the isotopes replacing the atoms are the same isotopes. Thus, in an embodiment, in case carbon atoms are replaced, all carbon atoms replaced are replaced by ¹³C atoms. In a further embodiment, at least 10%, in a further embodiment at least 20% in a further embodiment at least 30%, of the atoms of a specific element are replaced by a specific isotope. As the skilled person will understand, the above applies mutatis mutandis to fragments of the analyte and of the isotopologues which may be generated and detected during MS.

In an embodiment, the nucleophilic reagent and/or the side chain in the nucleophilic reagent-derivative of said lactone analyte derived from the nucleophilic reagent is/are isotopically labeled. Thus, e.g. in case the nucleophilic reagent is an amine, the atom replaced is at least one hydrogen and the isotope is ²H, the atom replaced is at least one carbon atom and the isotope is ¹³C, and/or the atom replaced is nitrogen and the isotope is ¹⁵N. In a further embodiment, all hydrogen atoms of the nucleophilic reagent and/or the side chain in the nucleophilic reagent-derivative of said lactone analyte derived from the nucleophilic reagent is/are replaced by ²H atoms. Thus, in an embodiment, e.g. in case the nucleophilic reagent is butylamine, the isotopically labelled derivative is butylamine-d9 (1-Aminobutane-d9, CAS No: 776285-22-4), and/or the nucleophilic reagent-derivative of the lactone analyte may be Everolimus-butylamide-d9, Everolimus-butylamide-d9-butylimine-d9, Tacrolimus-butylamide-d9, Tacrolimus-butylamide-d9-butylimine-d9, Sirolimus-butylamide-d9 and/or Sirolimus-butylamide-d9-butylimine-d9. As the skilled person is aware of, butylamine-d9 is the nomenclature for a 9fold ²H isotopomer of butylamine.

The present invention also relates to a calibration solution comprising a pre-determined concentration of a nucleophilic reagent-derivative of a lactone analyte, in an embodiment an immunomodulatory macrolide.

The term “calibration” is used herein in a broad sense in concurrence with typical use by the skilled person. Thus, the term calibration includes an operation which establishes under specified conditions a relation between quantity values obtained with measurement standards and corresponding quantity values of a calibrated instrument, i.e. a calibration sensu stricto. Calibration may, however, also be verification of measurement values. The term calibration further includes measures of adjusting or re-adjusting the calibrated instrument or its output to concur with the aforesaid quantity values obtained with measurement standards comprised in the internal calibrator, i.e. calibration in its usual, broader sense. Thus, in the method specified herein, the term calibration may also relate to the provision of a correlation of e.g. MS signals determined by an MS device with amounts of isotopologues comprised in a sample.

In accordance, the term “calibration solution”, as used herein, relates to a liquid composition comprising a predetermined amount of a nucleophilic reagent-derivative of a lactone analyte. As the skilled person will understand in view of the description herein, the nucleophilic reagent-derivative of a lactone analyte in an embodiment is selected such as to correspond to a derivative of a lactone analyte to be determined with a nucleophilic reagent used in the determining method to be used. Thus, in case e.g. tacrolimus shall be determined in a sample and the determining method comprises contacting said sample with n-butylamine, the calibration solution may in particular comprise a pre-determined amount of tacrolimus-butylamide-d9, e.g. in case of internal calibration, or of tacrolimus-butylamide, e.g. in case of external calibration. In an embodiment, the liquid composition comprises the nucleophilic reagent-derivative of a lactone analyte at a concentration as specified elsewhere herein for the lactone analytes, the indicated concentration ranges, in an embodiment, referring to the concentration of one lactone analyte. As will be understood, accordingly, the total sum of nucleophilic reagent-derivative of a lactone analyte in the preparation may exceed the referenced concentration ranges, e.g. in case the calibration solution comprises nucleophilic reagent-derivatives of more than one lactone analyte, e.g. of Everolimus, Sirolimus, and Tacrolimus. Also, the calibration solution may be a calibration stock solution for dilution to cover the expected range of lactone analyte concentrations. Thus, in an embodiment, the lactone analyte is an immunomodulatory macrolide, in an embodiment is Everolimus, Sirolimus or Tacrolimus. In a further embodiment, the nucleophilic reagent side chain is N-alkyl, in an embodiment is N-butyl. In an embodiment, the calibration solution is stable at a temperature of at most 10° C. for at least 2 weeks, in an embodiment at least four weeks, in a further embodiment at least eight weeks.

Furthermore, the present invention relates to a nucleophilic reagent-derivative of an immunomodulatory macrolide and/or a calibration solution as specified herein for use in diagnosis, in particular for use in diagnosing inappropriate dosage of said immunomodulatory macrolide in a sample of a subject.

The term “subject”, as used herein, relates to an animal, in an embodiment a mammal, in a further embodiment a primate, in a further embodiment a human. In an embodiment, the subject is an experimental animal, in particular a mouse, rat, guinea pig, pig, or dog. In a further embodiment, the subject is a livestock or companion animal, in particular a cat, dog, goat, sheep, cattle, horse, or pig. In an embodiment, the subject is a subject known or suspected to be treated with at least one immunomodulatory compound, in particular at least one immunomodulatory macrolide. Thus, in an embodiment, the subject is a transplant recipient, in particular a human transplant recipient under immunosuppressive treatment, in particular under immunomodulatory macrolide treatment.

The present invention also relates to a kit comprising (i) a nucleophilic reagent and a nucleophilic reagent-derivative of a lactone analyte, (ii) a calibration solution comprising a pre-determined concentration of a nucleophilic reagent-derivative of a lactone analyte, and/or (iii) a nucleophilic reagent-derivative of an immunomodulatory macrolide, all as specified herein above, in a housing.

The term “kit”, as used herein, refers to a collection of the aforementioned compounds, means or reagents which may or may not be packaged together. The components of the kit may be comprised by separate vials (i.e. as a kit of separate parts) or provided in a single vial, e.g. as a composition as specified herein above. The housing of the kit in an embodiment allows translocation of the compounds of the kit, in particular common translocation; thus, the housing may in particular be a transportable container comprising all specified components. Moreover, it is to be understood that the kit of the present invention may be used for practicing the methods referred to herein above. It is, in an embodiment, envisaged that all components are provided in a ready-to-use manner for practicing the methods referred to above. Further, the kit, in an embodiment, contains instructions for carrying out said methods. The instructions can be provided by a user's manual on paper or in electronic form. For example, the manual may comprise instructions for interpreting the results obtained when carrying out the aforementioned methods using the kit. As is understood by the skilled person, the nucleophilic reagent, the nucleophilic reagent-derivative of a lactone analyte comprised in the kit, including being comprised in a calibration solution, may be isotopically labelled as specified herein above. Thus, the kit may e.g. also comprise (iv) an isotopically labelled nucleophilic reagent and an isotopically labelled nucleophilic reagent-derivative of a lactone analyte, (v) an internal standard comprising a pre-determined concentration of an isotopically labelled nucleophilic reagent-derivative of a lactone analyte, in particular of a nucleophilic reagent-derivative of an immunomodulatory macrolide.

The present invention also relates to a device for determining a lactone analyte, said device comprising an analysis unit configured to determine a nucleophilic reagent-derivative of said lactone analyte and an evaluation unit configured to determine said lactone analyte based on the determination of the nucleophilic reagent-derivative by the analysis unit, wherein said device further comprises (i) a nucleophilic reagent and a nucleophilic reagent-derivative of a lactone analyte, (ii) a calibration solution comprising a pre-determined concentration of a nucleophilic reagent-derivative of a lactone analyte, and/or (iii) a nucleophilic reagent-derivative of an immunomodulatory macrolide as specified herein above.

The term “device”, as used herein, relates to a system comprising the aforementioned units operatively linked to each other to allow the diagnosis according to the methods of the invention. Preferred detection agents which can be used for the analyzing unit are disclosed elsewhere herein. The analyzing unit may, e.g. comprise detection agents in immobilized form on a solid support which is to be contacted to the sample comprising the lactone analyte the amount of which is to be determined. Moreover, the analyzing unit can also comprise a detector which determines the amount of detection agent which is specifically bound to the biomarker(s). The determined amount can be transmitted to the evaluation unit. Said evaluation unit comprises a data processing element, such as a computer, with an implemented algorithm for carrying out a calculation of ratios, a comparison of said calculated ratios and an evaluation of the result of the comparison by implementation of an computer-based algorithm carrying out the steps of the method of the present invention set forth elsewhere herein in detail. The results may be given as output of parametric diagnostic raw data. It is to be understood that these data will usually need interpretation by the clinician. However, also envisaged are expert system devices wherein the output comprises processed diagnostic raw data the interpretation of which does not require a specialized clinician. The device may in particular be configured to perform a method as described herein.

In an embodiment of the device, said analyzing unit comprises detection means for determining the amount of the nucleophilic reagent-derivative of said lactone analyte and an algorithm implemented in the evaluation unit comparing the value determined with at least one calibration and, optionally, a value measured from an internal standard, whereby the lactone analyte is determined. It follows from the above that portions of some steps of methods disclosed and described herein may be performed by one or more computing device(s). A computing device may be a general purpose computer or a portable computing device, for example. It should also be understood that multiple computing devices may be used together, such as over a network or other methods of transferring data, for performing one or more steps of the methods disclosed herein. Exemplary computing devices include desktop computers, laptop computers, personal data assistants (“PDA”), cellular devices, tablet computers, servers, and the like. In general, a computing device comprises a processor capable of executing a plurality of instructions (such as a program of software).

The device may in particular be a mass spectrometry device or comprise a mass spectrometry unit. The term “mass spectrometry device”, abbreviated as “MS device”, is understood by the skilled person. In an embodiment, the term relates to a device configured for performing a mass spectrometry (MS); thus, the device, in an embodiment, comprises at least one MS unit. As used herein, the term “mass spectrometry unit”, in an embodiment, relates to a mass analyzer configured for detecting at least one analyte based on a mass to charge ratio of the analyte or a fragment thereof. In an embodiment, the MS unit is a tandem mass spectrometry (MS/MS) unit, in a further embodiment a triple quadrupole MS (QqQ-MS), in a further embodiment in Multiple Reaction Monitoring (MRM) mode. The MS device or MS unit may further comprise at least one ionization source configured for generating molecular ions and for transferring the molecular ions into the gas phase. Ionization methods and appropriate ionization units are known in the art and include in particular electron ionization (EI), chemical ionization (CI), electrospray ionization (ESI), atmospheric pressure ionization (APCI), atmospheric pressure photoionization (APPI), and matrix assisted laser desorption/ionization (MALDI).

In an embodiment, the MS device or MS unit comprises a chromatography MS device, in particular a gas chromatography MS (GC-MS) device or a liquid chromatography MS (LC-MS) device, terms understood by the skilled person. Thus, in an embodiment, the device is configured for performing a combination of chromatography (e.g. LC or GC)) with mass spectrometry (MS). Thus, the device, in an embodiment, comprises at least one LC and/or GC unit, and at least one MS unit, wherein the LC and/or GC unit(s) and the MS unit are coupled via at least one interface. As used herein, the term “liquid chromatography (LC) unit”, in an embodiment, relates to an analytical module configured to separate one or more analytes of interest of a sample from other components of the sample via liquid chromatography, in an embodiment for detection of the one or more analytes with the mass spectrometry device. The LC may be based on any separation principle deemed appropriate by the skilled person; in an embodiment, the LC is reverse phase chromatography, hydrophobic interaction chromatography, ion exchange chromatography, size exclusion chromatography, affinity chromatography, or chiral chromatography; in a further embodiment, the LC is reverse phase chromatography. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analyte(s) of interest. The LC unit may be or may comprise at least one high-performance liquid chromatography (HPLC) unit and/or at least one micro liquid chromatography (uLC) device. The term “gas chromatography” is understood by the skilled person; in an embodiment the same separation principles as for LC are applicable, however, the mobile phase being a gas in GC.

The invention further discloses and proposes a computer program including computer-executable instructions for performing the method according to the present invention in one or more of the embodiments enclosed herein when the program is executed on a computer or computer network. Specifically, the computer program may be stored on a computer-readable data carrier. Thus, specifically, one, more than one or even all of method steps a) to d) as indicated above may be performed by using a computer or a computer network, in an embodiment by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

In an embodiment, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:

A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In view of the above, the following embodiments are particularly envisaged:

Embodiment 1: A method for determining a lactone analyte in a sample, comprising
(i) contacting said sample with a derivatization reagent comprising a nucleophilic reagent;
(ii) determining a nucleophilic reagent-derivative of said lactone analyte obtained in step (i), and
(iii) determining said lactone analyte based on the determination of the nucleophilic reagent-derivative of said lactone analyte in step (ii).

Embodiment 2: The method of embodiment 1, wherein said lactone analyte is a macrocyclic lactone comprising an at least five-membered lactone ring, in an embodiment is a macrolide.

Embodiment 3: The method of embodiment 1 or 2, wherein said lactone analyte is an immunomodulatory macrolide.

Embodiment 4: The method of any one of embodiments 1 to 3, wherein said lactone analyte is Everolimus, Sirolimus or Tacrolimus.

Embodiment 5: The method of any one of embodiments 1 to 4, wherein said nucleophilic reagent is an amine, an alcohol, a thiol, or a selenol.

Embodiment 6: The method of any one of embodiments 1 to 5, wherein said nucleophilic reagent is a primary amine, in an embodiment a primary alkyl amine, in a further embodiment a C1 to C8 alkyl primary amine.

Embodiment 7: The method of any one of embodiments 1 to 6, wherein said nucleophilic reagent is an n-alkyl primary amine, in an embodiment a C1 to C8 n-alkyl primary amine. Embodiment 8: The method of any one of embodiments 1 to 7, wherein said nucleophilic reagent is a butylamine.

Embodiment 9: The method of any one of embodiments 1 to 8, wherein said nucleophilic reagent is n-butylamine (CAS NO: 109-73-9).

Embodiment 10: The method of any one of embodiments 1 to 9, wherein the concentration of the nucleophilic reagent in step (i) is of from 0.1 M to 10 M, in an embodiment is of from 0.5 M to 1.5 M, in a further embodiment is about 1 M, in an embodiment is 1 M.

Embodiment 11: The method of any one of embodiments 1 to 10, wherein in step (i) the derivatization reagent comprising the nucleophilic reagent at a concentration of from 1 M to 10 M is contacted with said sample at a ratio of from 1:5 to 10:1, in an embodiment of from 1:2 to 5:1.

Embodiment 12: The method of any one of embodiments 1 to 11, wherein the pH during the contacting in step (i) is of from 4 to 10, in an embodiment of from 5 to 9, in a further embodiment of from 6 to 8, and/or wherein during all or part of step step (ii), the pH is of from 1 to 6, in an embodiment of from 2 to 5, in a further embodiment of from 3 to 4.

Embodiment 13: The method of any one of embodiments 1 to 12, wherein step (i) is performed in a time frame of less than 5 minutes, in an embodiment less than 2 minutes, in a further embodiment less than 1 minute.

Embodiment 14: The method of any one of embodiments 1 to 13, wherein said step (ii) comprises separating said nucleophilic reagent-derivative of said lactone analyte from sample matrix constituents, in an embodiment by chromatography, in a further embodiment liquid chromatography (LC) or gas chromatography (GC).

Embodiment 15: The method of any one of embodiments 1 to 14, wherein said step (ii) comprises mass spectrometry (MS).

Embodiment 16: The method of any one of embodiments 1 to 15, wherein said step (ii) comprises MS, MS/MS, LC-MS, or GC-MS, in an embodiment LC-MS, in a further embodiment LC-MS/MS.

Embodiment 17: The method of any one of embodiments 1 to 16, wherein the determining step (ii) comprises quantitatively determining a parameter directly proportional to the concentration of the lactone analyte and/or the nucleophilic reagent-derivative of said lactone analyte, in an embodiment to the nucleophilic reagent-derivative of said lactone analyte.

Embodiment 18: The method of any one of embodiments 1 to 17, wherein said step (iii) comprises comparing the value of the parameter directly proportional to the concentration of the lactone analyte and/or the nucleophilic reagent-derivative of said lactone analyte obtained in step (ii) to a value of a corresponding parameter obtained for at least one calibration sample and/or to a value of a corresponding parameter obtained for said internal standard.

Embodiment 19: The method of any one of embodiments 1 to 18, wherein said step (iii) comprises calculating an amount or concentration of the lactone analyte in the sample.

Embodiment 20: The method of any one of embodiments 1 to 19, wherein said sample is a biological sample, in an embodiment comprising biological macromolecules.

Embodiment 21: The method of any one of embodiments 1 to 20, wherein said sample is a sample of blood, serum, or plasma

Embodiment 22: The method of any one of embodiments 1 to 21, wherein said sample is a sample comprising cells.

Embodiment 23: The method of any one of embodiments 1 to 22, wherein said sample is a blood sample Embodiment 24: The method of any one of embodiments 1 to 23, wherein said sample is a sample comprising cells and wherein said method further comprises a step of lysing cells preceding step (ii).

Embodiment 25: The method of embodiment 24, wherein said step of lysing cells is performed concomitant to step (i).

Embodiment 26: The method of embodiment 24 or 25, wherein said lysing cells comprises contacting said sample with a lysis reagent.

Embodiment 27: The method of embodiment 26, wherein said lysis reagent is said derivatization reagent.

Embodiment 28: The method of any one of embodiments 1 to 27, wherein said method further comprises adding an internal standard to said sample before step (ii), in an embodiment before step (i).

Embodiment 29: The method of embodiment 28, wherein said internal standard comprises an isotope-labeled derivative of the lactone analyte and/or of the nucleophilic reagent-derivative of said lactone analyte.

Embodiment 30: The method of embodiment 28 or 29, wherein said internal standard comprises an isotope-labeled derivative of the lactone analyte and/or of the nucleophilic reagent-derivative of said lactone analyte, in an embodiment a nucleophilic reagent-derivative of an immunomodulatory macrolide according to any one of embodiments 47 to 49.

Embodiment 31: The method of any one of embodiments 28 to 30, wherein said internal standard comprises an isotope-labeled butylamide derivative of the lactone analyte.

Embodiment 32: The method of any one of embodiments 28 to 31, wherein said internal standard comprises, in an embodiment is, an isotope-labeled n-butylamide derivative of Everolimus, Sirolimus and/or Tacrolimus.

Embodiment 33: The method of any one of embodiments 28 to 32, wherein said internal standard comprises, in an embodiment is, Everolimus-butylamide-d9, Sirolimus-butylamide-d9, and/or Tacrolimus-butylamide-d9.

Embodiment 34: The method of any one of embodiments 28 to 33, wherein the determining step (ii) further comprises quantitatively determining a parameter directly proportional to the concentration of the isotope-labeled derivative of the lactone analyte and/or of the nucleophilic reagent-derivative of said lactone analyte.

Embodiment 35: The method of any one of embodiments 1 to 34, wherein said method further comprises enriching the lactone analyte and/or the nucleophilic reagent-derivative of said lactone analyte before step (ii), in an embodiment by binding to a solid surface.

Embodiment 36: The method of any one of embodiments 1 to 35, wherein said step (i) is performed at most 3 h after the sample is obtained, in an embodiment at most 10 min after the sample is obtained.

Embodiment 37: The method of any one of embodiments 1 to 36, wherein said method further comprises obtaining a calibration curve based on at least two calibration samples comprising two pre-determined and non-identical concentrations of said nucleophilic reagent-derivative of said lactone analyte.

Embodiment 38: The method of any one of embodiments 1 to 37, wherein said method comprises removing insoluble sample constituents before step (ii), in an embodiment by centrifugation and/or filtration.

Embodiment 39: The method of any one of embodiments 1 to 38, wherein said sample is a biological sample comprising cells, in an embodiment is a blood sample, and wherein step (i) comprises contacting said sample with a derivatization reagent comprising a nucleophilic reagent and thereby lysing cells comprised in said sample.

Embodiment 40: The method of embodiment 39, wherein said nucleophilic reagent is butylamine, propylamine, pentylamine, or hexylamine, in an embodiment n-butylamine, n-propylamine, n-pentylamine, or n-hexylamine.

Embodiment 41: The method embodiment 39 or 40, wherein said method does not comprise a step of removing insoluble sample constituents in and between steps (i) and step (ii).

Embodiment 42: A method of lysing cells in a biological sample, comprising contacting said biological sample with a nucleophilic reagent, in an embodiment as specified in any one of embodiments 5 to 9, more preferably as specified in embodiment 40.

Embodiment 43: The method of embodiment 42, wherein said sample is a blood sample.

Embodiment 44: Use of a nucleophilic reagent for determining a lactone analyte in a sample, in an embodiment according to the method according to any one of embodiments 1 to 41.

Embodiment 45: Use of a nucleophilic reagent for lysing cells in a biological sample.

Embodiment 46: The use of embodiment 44 or 45, wherein said nucleophilic reagent is a nucleophilic reagent as specified in any one of embodiments 5 to 9.

Embodiment 47: A nucleophilic reagent-derivative of an immunomodulatory macrolide.

Embodiment 48: The nucleophilic reagent-derivative of an immunomodulatory macrolide of embodiment 47 having a structure of any one of formulas (I) to (IX), as specified herein in the description.

Embodiment 49: The nucleophilic reagent-derivative of an immunomodulatory macrolide of embodiment 47 or 48, wherein said immunomodulatory macrolide and/or the nucleophilic reagent side chain is/are isotopically labeled.

Embodiment 50: A calibration solution comprising a pre-determined concentration of a nucleophilic reagent-derivative of a lactone analyte, in an embodiment an immunomodulatory macrolide, in an embodiment wherein said calibration solution is stable at a temperature of at most 10° C. for at least 2 weeks, in an embodiment at least four weeks, in a further embodiment at least eight weeks.

Embodiment 51: The nucleophilic reagent-derivative of an immunomodulatory macrolide of any one of embodiments 47 to 49 or the calibration solution of embodiment 50, wherein said lactone analyte is an immunomodulatory macrolide, in an embodiment is Everolimus, Sirolimus or Tacrolimus.

Embodiment 52: The nucleophilic reagent-derivative of an immunomodulatory macrolide of any one of embodiments 47 to 49 or 51 or the calibration solution of embodiment 50 or 51, wherein said nucleophilic reagent side chain is N-alkyl, in an embodiment is N-butyl.

Embodiment 53: A nucleophilic reagent-derivative of an immunomodulatory macrolide and/or a calibration solution according to any one of embodiments 47 to 52, for use in diagnosis.

Embodiment 54: A nucleophilic reagent-derivative of an immunomodulatory macrolide and/or a calibration solution according to any one of embodiments 47 to 52, for use in diagnosing inappropriate dosage of said immunomodulatory macrolide in a sample of a subject.

Embodiment 55: A kit comprising (i) a nucleophilic reagent and a nucleophilic reagent-derivative of a lactone analyte, (ii) a calibration solution comprising a pre-determined concentration of a nucleophilic reagent-derivative of a lactone analyte, and/or (iii) a nucleophilic reagent-derivative of an immunomodulatory macrolide according to any one of embodiments 47 to 49, 51, and 52, in a housing.

Embodiment 56: A device for determining a lactone analyte comprising an analysis unit configured to determine a nucleophilic reagent-derivative of said lactone analyte and an evaluation unit configured to determine said lactone analyte based on the determination of the nucleophilic reagent-derivative by the analysis unit, wherein said device further comprises (i) a nucleophilic reagent and a nucleophilic reagent-derivative of a lactone analyte, (ii) a calibration solution comprising a pre-determined concentration of a nucleophilic reagent-derivative of a lactone analyte, and/or (iii) a nucleophilic reagent-derivative of an immunomodulatory macrolide according to any one of embodiments 47 to 49, 51, and 52.

Embodiment 57: The device of embodiment 56 configured to perform the method according to any one of embodiments 1 to 41 and/or the method according to any one of embodiments 42 or 43.

All references cited in this specification are herewith incorporated by reference with respect to their entire disclosure content and the disclosure content specifically mentioned in this specification.

### Figure Legends

FIG. 1: Derivatization reactions of Everolimus with n-butylamine; [1]: Everolimus; [4] and Everolimus-butylamide; [5] Everolimus-butylamide-butylimide, [16] Everolimus-butylamide-ketone.

FIG. 2: Chromatograms of product [5] of FIG. 1 at 19 seconds, monitored at two different MRM transitions (A and B). Chromatograms obtained after hemolysis/derivatization and workup of [1] in whole blood.

FIG. 3: Calibration Curve of product [5] of FIG. 1; MRM transition 1087-300.

FIG. 4: Calibration Curve of product [5] of FIG. 5; MRM transition 1087-1055.

FIG. 5: % recovery of target value obtained for four spiked whole blood samples at two different MRM transitions.

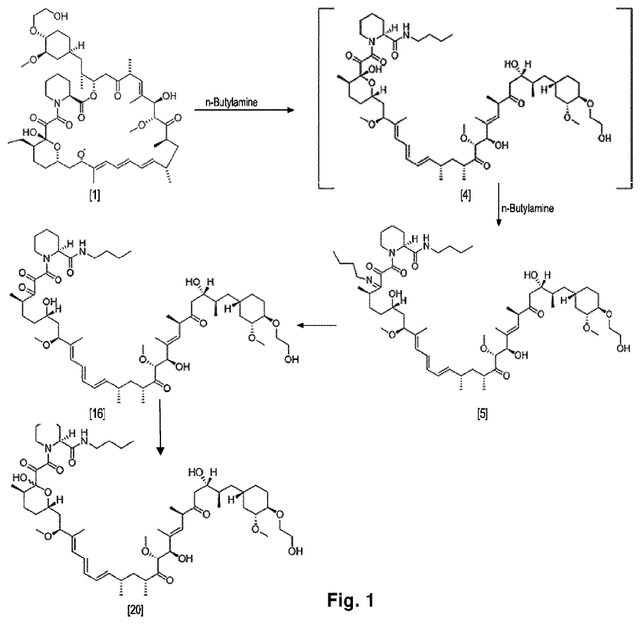

The following Examples shall merely illustrate the invention. They shall not be construed, whatsoever, to limit the scope of the invention. The reference number refer to the reaction scheme of FIG. 1.

### EXAMPLE 1: EXPERIMENTAL DESIGN

Whole blood samples containing [1] in a concentration range of 0.5-20 μg/mL as well as a hemolysis reagent containing n-butylamine (5.5 M in water). All investigations were performed on a fully automatic analyzer capable of purifying and measuring the products. Further, to establish the validity of the method, four spiked samples with known concentrations of [1] were measured and compared to the theoretical target values. Using a calibration curve of spiked [1] in whole blood.

### EXAMPLE 2: MATERIALS

For hemolysis, n-butylamine was diluted to a concentration of 5.5 M with water. Internal standard (ISTD) solution was prepared by mixing of Everolimus-¹³CH₂²H₄ in CH₃CN to obtain a 1 mg/mL solution. For HPLC, a C-18 2.6 μM, 1.0×50 mm column was used. An analyzer with automatic sample preparation and LC-MS Analyzer was used for sample preparation and measurement. LC-Solvent A: water, Solvent B: CH₃CN. The gradient is provided in Table 2.

### EXAMPLE 3: METHODS

Hemolysis and Derivatization Method: To Calibration samples and patient samples containing [1] (500 μL), ISTD Solution (5 μL) hemolysis reagent (500 μL) was added. These were placed on an analyzer, capable of performing multiple steps, prior to measurement of the compounds of interest via LC-MS/MS.

The protocol to measure [5] was as follows: To the sample (200 μL) magnetic beads (50 mg/mL, 40 μL) was added. Next, a magnetic force was applied to the sides of the vessel.

Subsequently, the supernatant is removed and the beads are washed twice, at the first step with water/CH₃CN (90/10, v/v, 150 μL) and at the second step with water/CH₃CN/NH₃ (50 mM), (60/10/30, v/v/v, 150 μL). Then, an elution mixture (CH₃CN/NH₃ (50 mM), 70/30, v/v) (80 μL) was added. Following incubation, again a magnetic force was applied and 50 μL of the supernatant is transferred to a new vessel. Of this mixture, 19 μL was then injected onto a HPLC column with a flow of 440 μL/min, and connected to a MS/MS measuring device. Precursor and product m/z, as well as the settings are found in Table 1.

TABLE 1
Precursor and product masses for MS/MS

| | | Time | | |
|---|---|---|---|---|
| Q1 | Q3 | (msec) | Transition name | CE |
| 1086.661 | 300.2 | 10 | custom_1086.661_300.2 | 49 |
| 1092.661 | 306.2 | 10 | custom_1092.661_306.2 | 49 |
| 1086.661 | 1054.7 | 10 | custom_1086.661_1054.7 | 33 |
| 1092.661 | 1060.7 | 10 | custom_1092.661_1060.7 | 33 |

TABLE 2
LC-gradient.

| Time [sec] | Solvent A [%] | Solvent B [%] |
|---|---|---|
| −36 | 70 | 30 |
| 0 | 70 | 30 |
| 36 | 2 | 98 |
| 72 | 2 | 98 |

### EXAMPLE 4: RESULTS

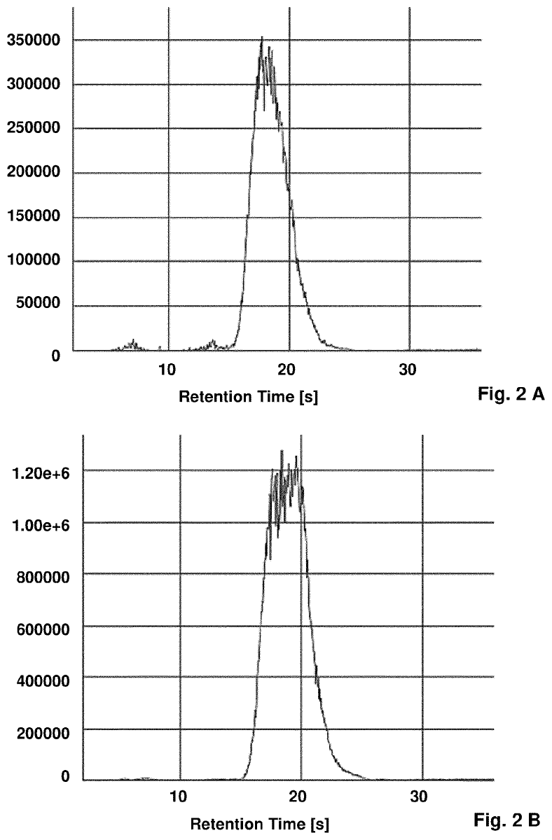

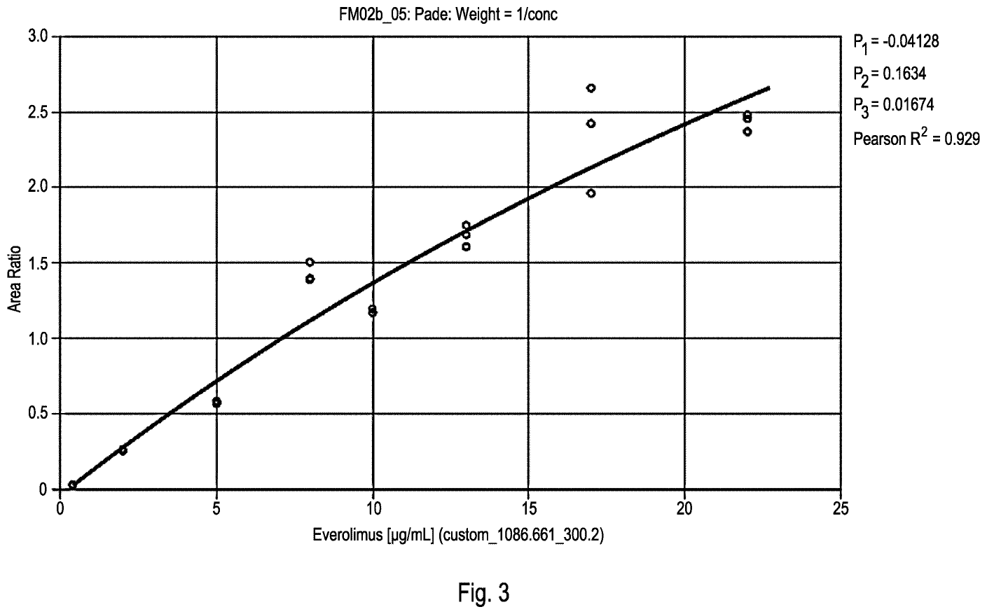

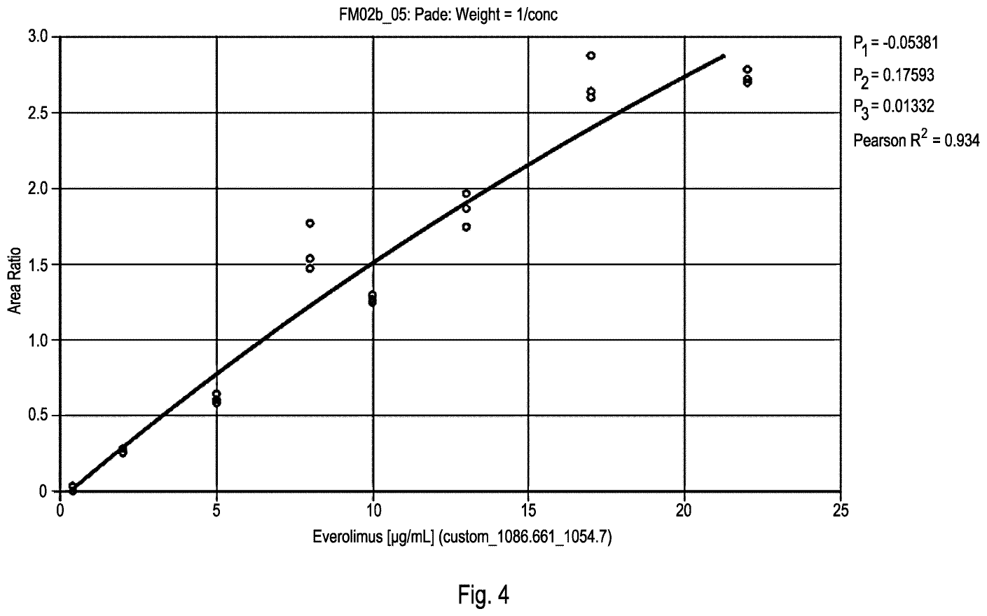

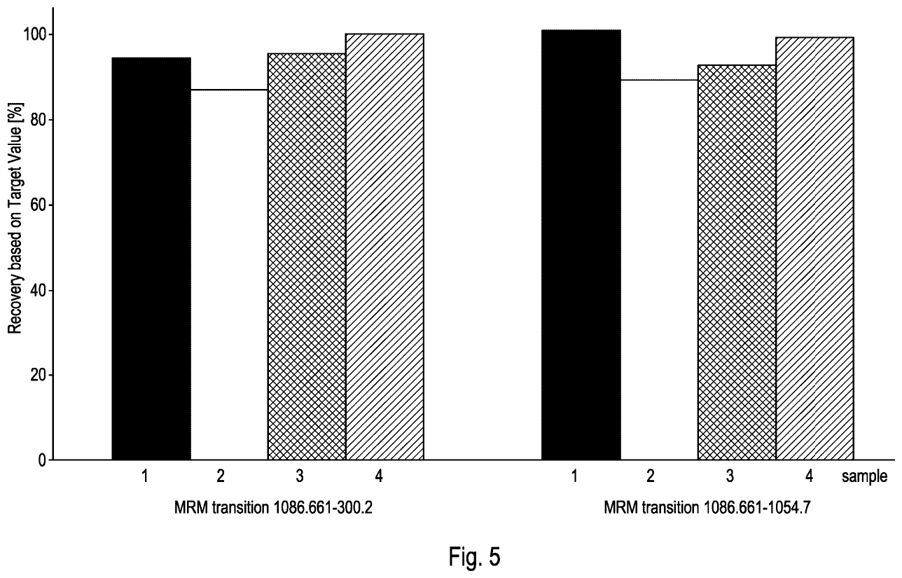

Measuring double derivatized compound [5], using two different MRM transitions, two chromatograms were obtained from a whole blood sample (spiked with [1]), presented in FIG. 2. For each MRM only one peak is visible. In term of quantitation of these peaks and their correlation to the theoretical target value, we obtained a calibration curve that we was used to quantify spiked whole blood samples with known concentrations of [1] (see FIGS. 3 and 4). Using these calibration curves, the concentration of [1] in the spiked whole blood samples was obtained. Of these obtained values the recovery (in %) to the theoretical target values was were calculated. The results are summarized in Tables 3a and b, or for a graphic overview in FIG. 5. The results show the validity of the described method in quantifying Everolimus in Immunosuppressant Drug Monitoring.

TABLE 3a

| | | MRM transition 1086.661-300.2 | | |
|---|---|---|---|---|
| | | | Recovery | |
| | Target | | based on | |
| Sample | Value | Concentration | Target Value | CV |
| ID | [μg/mL] | Value [μg/mL] | [%] | [%] |
| QC1 | 0.5 | 0.47 | 94.17 | 4.78 |
| QC2 | 1.5 | 1.31 | 87.01 | 1.70 |
| QC3 | 10 | 9.55 | 95.49 | 3.12 |
| QC4 | 20 | 20.03 | 100.14 | 3.31 |

TABLE 3b

| | | MRM transition 1086.661-1054.7 | | |
|---|---|---|---|---|
| | | | Recovery | |
| | Target | | based on | |
| Sample | Value | Concentration | Target Value | CV |
| ID | [μg/mL] | Value [μg/mL] | [%] | [%] |
| QC1 | 0.5 | 0.51 | 101.07 | 1.83 |
| QC2 | 1.5 | 1.34 | 89.38 | 11.68 |
| QC3 | 10 | 9.27 | 92.72 | 6.50 |
| QC4 | 20 | 19.84 | 99.19 | 0.32 |

### REFERENCES

EP 2 402 350 A1

The invention claimed is:

1. A method for determining a lactone analyte in a sample, comprising:
(i) contacting said sample with a derivatization reagent comprising a nucleophilic reagent;
(ii) determining a nucleophilic reagent-derivative of said lactone analyte obtained in step (i); and
(iii) determining said lactone analyte based on the determination of the nucleophilic reagent-derivative of said lactone analyte in step (ii).

2. The method of claim 1, wherein said lactone analyte is a macrocyclic lactone comprising an at least five-membered lactone ring embod.

3. The method of claim 1, wherein said lactone analyte is an immunomodulatory macrolide.

4. The method of claim 1, wherein said nucleophilic reagent is an amine, an alcohol, a thiol, or a selenol.

5. The method of claim 1, wherein said nucleophilic reagent is n-butylamine (CAS NO: 109-73-9).

6. The method of claim 1, wherein the concentration of the nucleophilic reagent in step (i) is of from 0.1 M to 10 M.

7. The method of claim 1, wherein said step (ii) comprises MS, MS/MS, LC-MS, or GC-MS.

8. The method of claim 1, wherein said method further comprises obtaining a calibration curve based on at least two calibration samples comprising two pre-determined and non-identical concentrations of said nucleophilic reagent-derivative of said lactone analyte.

9. A method of lysing cells in a biological sample, comprising contacting said biological sample with a nucleophilic reagent as specified in claim 4.

10. The method of claim 1, wherein said sample is a sample of blood, serum, or plasma.

11. (canceled)

12. A nucleophilic reagent-derivative of an immunomodulatory macrolide having a structure of any one of formulas (I) to (IX) 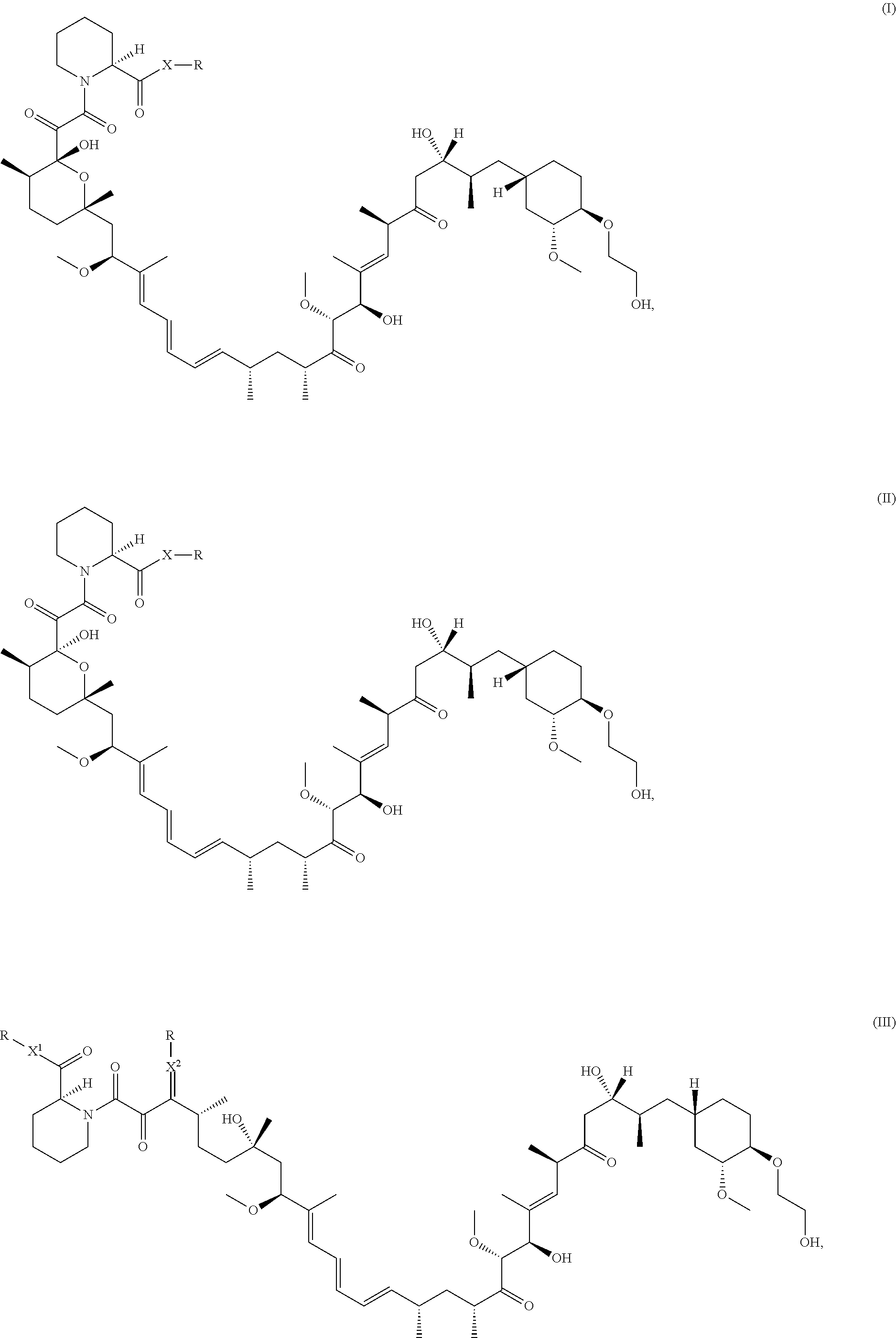 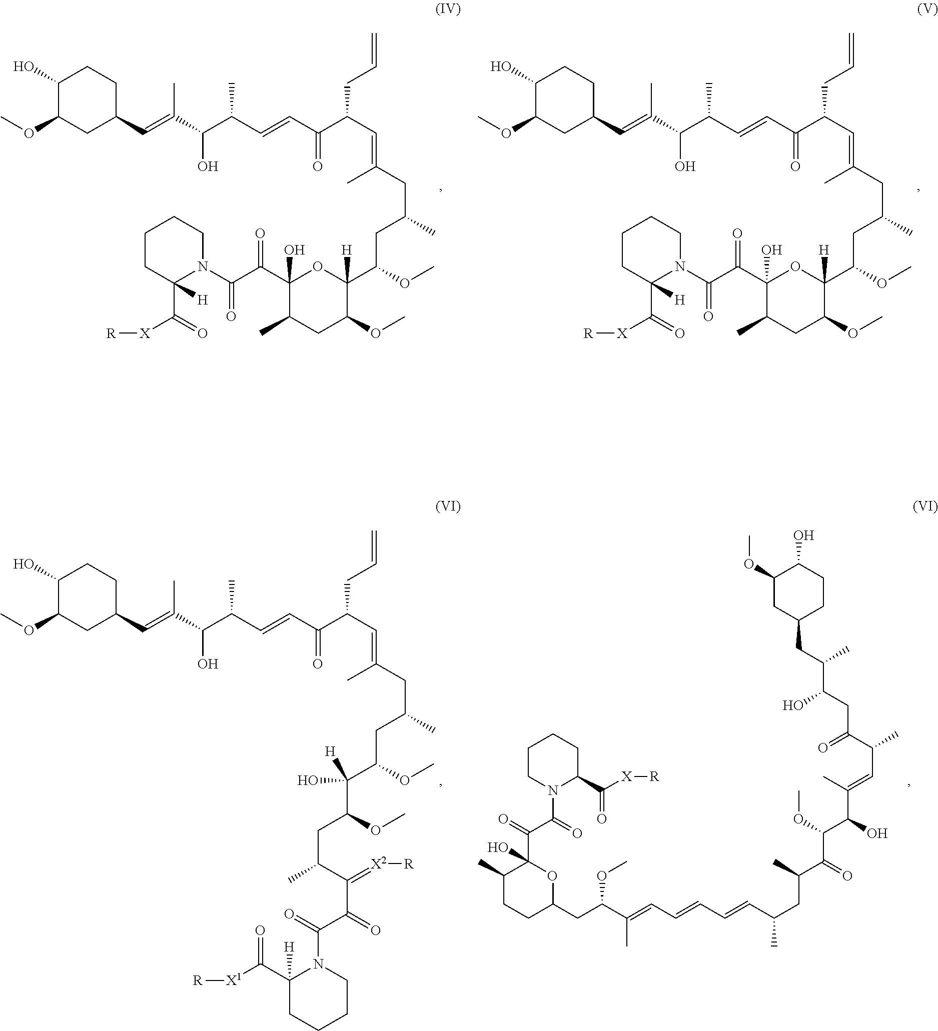 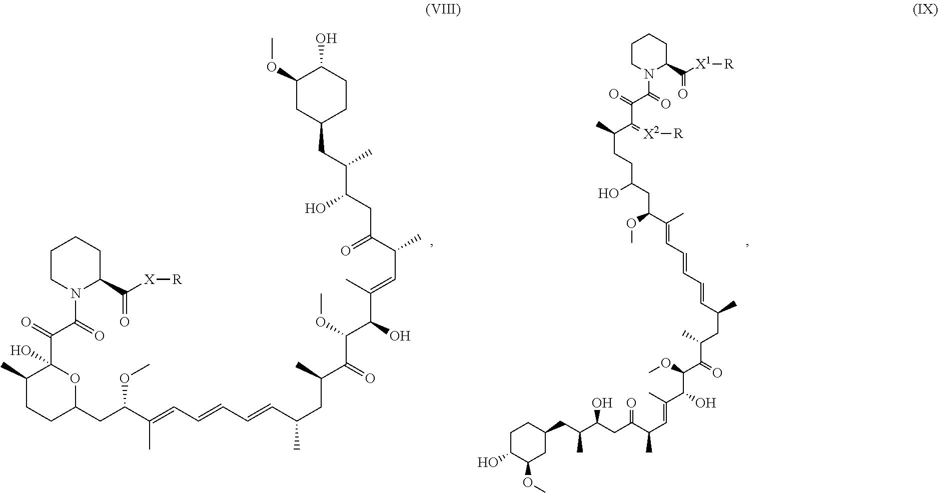
wherein in Formulas (I), (II), (IV), (V), (VII), and (VIII);
X is selected from NR^{a}, O, S, and Se, wherein R^{a} is selected from H and alkyl;
R is selected from H and alkyl;
and wherein in Formulas (III), (VI), and (IX):
X¹ is NR^{a} wherein R^{a} is selected from H and alkyl;
X² is N⁺R^{a}, wherein R^{a} is selected from H and alkyl; and
R is selected from alkyl.

13. The nucleophilic reagent-derivative of an immunomodulatory macrolide of claim 12, wherein said nucleophilic reagent side chain and/or the immunomodulatory macrolide is/are isotopically labeled.

14. A kit comprising (i) a nucleophilic reagent and a nucleophilic reagent-derivative of a lactone analyte, (ii) a calibration solution comprising a pre-determined concentration of a nucleophilic reagent-derivative of a lactone analyte, and/or (iii) a nucleophilic reagent-derivative of an immunomodulatory macrolide according to claim 12, in a housing.

15. A device for determining a lactone analyte comprising an analysis unit configured to determine a nucleophilic reagent-derivative of said lactone analyte and an evaluation unit configured to determine said lactone analyte based on the determination of the nucleophilic reagent-derivative by the analysis unit, wherein said device further comprises (i) a nucleophilic reagent and a nucleophilic reagent-derivative of a lactone analyte, (ii) a calibration solution comprising a pre-determined concentration of a nucleophilic reagent-derivative of a lactone analyte, and/or (iii) a nucleophilic reagent-derivative of an immunomodulatory macrolide according to claim 12.

16. The method of claim 2, wherein the macrocyclic lactone comprising an at least five-membered lactone ring is a macrolide.

17. The method of claim 3, wherein the immunomodulatory macrolide is Everolimus, Sirolimus or Tacrolimus.

18. The method of claim 4, wherein said nucleophilic reagent is a primary amine.

19. The method of claim 6, wherein the concentration of the nucleophilic reagent in step (i) is of from 0.5 M to 1.5 M, is about 1 M, or is 1 M.

20. The method of claim 10, wherein said sample is a sample of blood.
